(19) United States
(12) Patent Application Publication
(10) Pub. No.: US 2017/0325693 A1
(43) Pub. Date: Nov. 16, 2017

(54) PHOTOACOUSTIC APPARATUS AND CONTROL METHOD OF PHOTOACOUSTIC APPARATUS

(71) Applicant: CANON KABUSHIKI KAISHA, Tokyo (JP)

(72) Inventors: Tatsuki Fukui, Yokohama-shi (JP); Atsushi Takahashi, Ebina-shi (JP); Satoshi Yuasa, Yokohama-shi (JP); Kazuhiko Fukutani, Yokohama-shi (JP)

(21) Appl. No.: 15/584,163

(22) Filed: May 2, 2017

(30) Foreign Application Priority Data
May 10, 2016 (JP) 2016-094564

(51) Int. Cl. A61B 5/00; A61B 5/026

(52) U.S. Cl. CPC A61B 5/0095; A61B 5/026; A61B 5/74

(57) ABSTRACT

A photoacoustic apparatus, comprises a light source that irradiates an object with light; a plurality of acoustic wave detectors that receive acoustic waves generated from the object, convert the acoustic waves into an electrical signal, and output the electrical signal; and an information acquisition unit that acquires information of the object, based on the electrical signals, wherein the information acquisition unit acquires, for each of the acoustic wave detectors, a change in the intensity of the electrical signal, after the object that has been injected with a contrast agent is irradiated with light that decomposes the contrast agent, and acquires information relating to blood flow, based on the change in the intensity of the electrical signal.

### BACKGROUND OF THE INVENTION

### Field of the Invention

The present invention relates to a photoacoustic apparatus for acquiring information on the interior of an object.

### Description of the Related Art

Ongoing research is being conducted, in the medical field, on technologies for imaging morphological information and functional information on the interior of an object. Photoacoustic tomography (PAT) has been proposed in recent years as one such technology.

In PAT a living body as the object is irradiated with light such as pulsed laser light, and acoustic waves (typically ultrasonic waves) are generated thereupon as the light is absorbed by living tissue inside the object. This phenomenon is referred to as the photoacoustic effect, and the acoustic waves generated on account of the photoacoustic effect are referred to as photoacoustic waves. Absorptivity towards light energy varies depending on the tissue that makes up the object, and accordingly the sound pressure of the photoacoustic waves that are generated varies as well. In PAT, the generated photoacoustic waves are received by a probe, and a received signal is analyzed mathematically; as a result, it becomes possible to obtain information on the interior of the object, for instance initial sound pressure, optical characteristic values (in particular, light energy absorption density and absorption coefficients), as well as three-dimensional distributions of the foregoing. Further, PAT can be used for instance for identifying a distribution of an absorber within a living body, and for pinpointing the location of a malignant tumor.

In a photoacoustic measurement, an initial sound pressure P0 of acoustic waves generated by a light absorber within the object can be expressed by the following equation.

P0=Γ·μa·Φ

Herein, Γ is the Gruneisen coefficient, resulting from dividing the product of the coefficient of volumetric expansion β and the square speed of sound c by the specific heat capacity Cp at constant pressure. As is known, Γ takes on a substantially constant value if the object is determined. Further, μa is the absorption coefficient of the light absorber, and Φ is the quantity of light (also referred to as light fluence) that reaches the light absorber.

The acoustic waves generated by the light absorber propagate within the object are received by a probe that is disposed on the surface of the object. The change with time in the sound pressure of the received acoustic waves is measured, whereupon the initial sound pressure distribution P0 can be calculated by applying an image reconstruction method such as a back-projection method to the measurement result. A light energy density distribution or absorption coefficient distribution can also be obtained on the basis of the initial sound pressure distribution P0.

As is known, by injecting into an object a contrast agent in the form of a light absorber having a known optical characteristic towards the light that is irradiated it becomes possible thereto acquire acoustic waves; according to the abundance of the contrast agent.

A technology referred to as photoacoustic microscopy also exists that involves acquiring two-dimensional information by relying on the photoacoustic effect. When acquiring two-dimensional information, measurements can be performed in a shorter time than in the case of photoacoustic tomography, and various types of functional imaging can be performed that are not possible in photoacoustic tomography. For instance, the Journal of Biomedical Optics 12(6), 064006 November/December 2007 discloses a technology for visualizing blood flow velocity using a blood vessel model.

However, it is a feature of photoacoustic tomography that measurements characteristically take time, in order to detect acoustic waves generated by sound sources, through a probe disposed around the object, and acquire a three-dimensional distribution of sound sources as a result of an image reconstruction process. It has accordingly been difficult to acquire information, such as the direction and speed of blood flow, with changes over time.

It is an object of the present invention, arrived at in the light of the above technical problems of conventional art, to achieve acquisition of information pertaining to blood flow in photoacoustic tomography.

### SUMMARY OF THE INVENTION

The present invention in its one aspect provides a photoacoustic apparatus, comprising a light source that irradiates an object with light; a plurality of acoustic wave detectors that receive acoustic waves generated from the object due to the light, convert the acoustic waves into an electrical signal, and output the electrical signal; and an information acquisition unit that acquires information on the interior of the object, on the basis of the electrical signals, wherein the information acquisition unit acquires, for each of the acoustic wave detectors, a change in the intensity of the electrical signal, after the object that has been injected with a contrast agent is irradiated with light that decomposes the contrast agent, and acquires information relating to blood flow inside the object, on the basis of the change in the intensity of the electrical signal.

The present invention in its another aspect provides a method for controlling a photoacoustic apparatus having a light source that irradiates an object with light, and a plurality of acoustic wave detectors that receive an acoustic wave generated within the object on account of the light, and convert the acoustic wave into an electrical signal, the method comprising a first information acquisition step of acquiring information on the interior of the object, on the basis of the electrical signal; and a second information acquisition step of acquiring, for each of the acoustic wave detectors, a change in the intensity of the electrical signal, after the object that has been injected with a contrast agent is irradiated with light that decomposes the contrast agent, and acquiring information relating to blood flow inside the object, on the basis of a change in the intensity of the electrical signal.

Further features of the present invention will become apparent from the following description of exemplary embodiments (with reference to the attached drawings).

### BRIEF DESCRIPTION OF THE DRAWINGS

FIG. 1 is a system configuration diagram of a photoacoustic apparatus according to a first embodiment;

FIG. 2 is a flowchart diagram of a process executed by the photoacoustic apparatus according to the first embodiment;

FIG. 3 is a system configuration diagram of a photoacoustic apparatus according to a fifth embodiment; and

FIG. 4 is a plan-view diagram of a probe and an irradiation unit according to the fifth embodiment.

### DESCRIPTION OF THE EMBODIMENTS

Embodiments of the present invention will be explained in detail below with reference to accompanying drawings. Identical constituent elements will in principle be denoted by the same reference numerals, and an explanation thereof will be omitted. The dimensions, materials and shapes of constituent parts used in the explanation of the embodiments, relative positions between these constituent parts, and other features, are to be modified as appropriate in accordance with the configuration of the equipment to which the present invention is to be applied, and in accordance with various other conditions, and thus do not constitute features that limit the scope of the invention.

### First Embodiment

The photoacoustic apparatus according to the present embodiment is an apparatus for visualizing i.e. for imaging characteristic information pertaining to optical characteristics of the interior of an object, through irradiation of the object with pulsed light and through reception and analysis of photoacoustic waves generated inside the object on account of the pulsed light.

The term characteristic information relating to optical characteristics denotes ordinarily a generation source distribution of acoustic waves within the object, an initial sound pressure distribution, a light absorption energy density distribution, an absorption coefficient distribution, as well as a characteristic distribution related to the concentration of a tissue-constituting substance. Characteristic distributions related to concentration include for instance distributions of an oxygen saturation degree, of a value resulting from weighting an oxygen saturation degree by the magnitude of an absorption coefficient or the like, of total hemoglobin concentration, of oxyhemoglobin concentration of deoxyhemoglobin concentration. The characteristic distribution may be a distribution of glucose concentration, collagen concentration, or melanin concentration, or volume fractions of fat and water.

Characteristic information at a plurality of positions may be acquired in the form of a two-dimensional or three-dimensional characteristic information distribution. The characteristic distribution is generated in the form of image data that denotes characteristic information on the interior of the object. Characteristic information on the interior of the object is also referred to as object information.

In the present embodiment the term acoustic waves denotes typically ultrasonic waves, and includes elastic waves referred to as sound waves, ultrasonic waves, acoustic waves, photoacoustic waves and photoultrasonic waves. Acoustic waves generated on account of the photoacoustic effect are referred to as photoacoustic waves or photoultrasonic waves. In the present embodiment, the term light encompasses electromagnetic waves such as visible light rays and infrared rays. The apparatus can select as appropriate light of a specific wavelength, depending on the components to be measured.

<Problems in the Related Art>

A given measurement time is required in many cases, since photoacoustic measurement is a method that involves estimating the position of a sound source that is present within the object through analysis of photoacoustic waves received by the probe.

For instance, it is necessary to repeatedly perform, a plurality of times, a series of operations that involve irradiating the object with light and receiving acoustic waves, in a state where the probe is fixed at a given position.

The most ideal implementation of photoacoustic measurement involves arranging probes in all directions, in 360 degrees surrounding the object, and receiving thereupon the generated acoustic waves. However, it is extremely difficult to arrange a probe in all directions relative to the object, on account of the shape and structure of the object and due to spatial constraints. Therefore, a method is often resorted to that involves moving a probe disposed around an object to a plurality of reception positions, and receiving acoustic waves at each reception position. When resorting to this method it is necessary to reconstruct information after reception of receive acoustic waves at the plurality of reception positions while moving the probe, to generate an image corresponding to characteristic information on the interior of the object. Accordingly, a given lapse of time is required until that characteristic information is generated.

That is, conventional photoacoustic apparatuses have problems in that although the apparatuses are capable of imaging sites at which blood is present, but the apparatuses are not capable of acquiring information such as a blood flow direction and a flow rate changing over time.

<System Configuration>

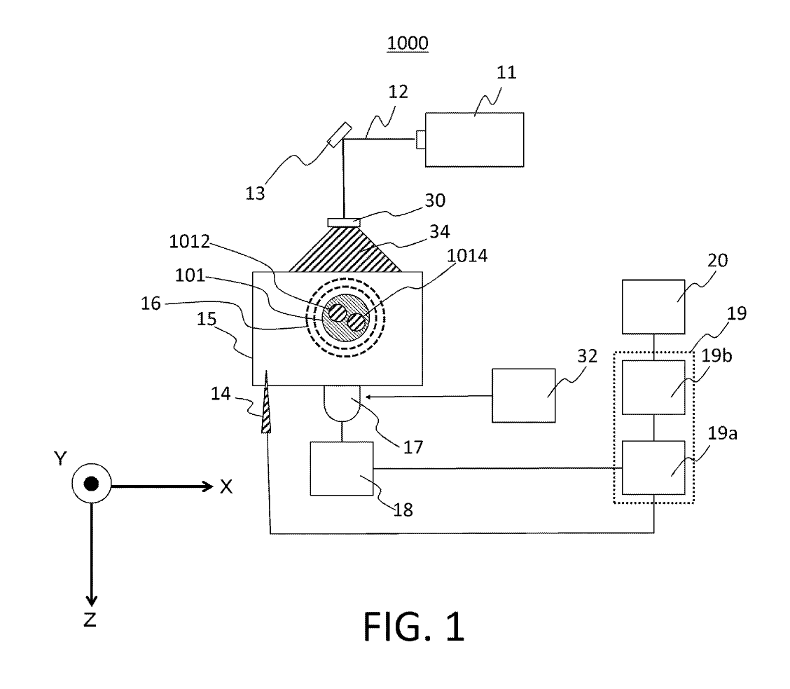

An explanation follows next on the configuration of a photoacoustic apparatus according to the first embodiment, for solving the above-described problems. FIG. 1 is a block configuration diagram of a photoacoustic apparatus 1000 according to the first embodiment.

The photoacoustic apparatus 1000 is an apparatus for acquiring, as viewable images, object information in the form of an optical characteristic value on the interior of the object. The photoacoustic apparatus 1000 according to the present embodiment has a light source 11, an optical system 13, an injection unit 14, a probe 17, a signal collecting unit 18, a signal processing unit 19 and an input/output unit 20. The various means that make up the photoacoustic apparatus according to the present embodiment will be explained next.

The explanation of the present embodiment will deal with three types of light absorber as imaging targets, namely a contrast agent (reference symbol 1012) being an artificial light absorber, a non-artificial absorber (reference symbol 1014) being a light absorber other than a contrast agent, and a combined absorber (reference symbol 101) being a light absorber that combines the foregoing two absorbers.

<<Light Source 11>>

The light source 11 is a means for emitting laser light (pulsed light) of a specific wavelength is absorbed by a specific component (for instance, blood) that makes up a living body as object.

The light source is preferably a laser light source in order to achieve a large output, but a light-emitting diode, a flash lamp or the like can be used instead of a laser. A solid-state laser, a gas laser, a dye laser, a semiconductor laser or the like can be used in a case where a laser is utilized as the light source. The timing, waveform, intensity and so forth of irradiation are controlled by a light source control means, not shown. The light source control means may be integrated with the light source.

The wavelength of pulsed light is a specific wavelength absorbed by a specific component, from among the components that make up the object, and is preferably a wavelength at which light propagates up to the interior of the object. Specifically, the wavelength lies preferably in the range from at least 700 nm to not more than 1100 nm in a case where the object is a living body. To work out an optical characteristic value distribution of living tissue at a position comparatively close to the surface of the living body, there may be used a wavelength in a range wider than the above wavelength region, for instance a wavelength in the range of 400 nm to 1600 nm. In a case in particular where the object 15 is a living body, it is preferable to use a wavelength in the near-infrared region, from 700 nm to 900 nm, being a safe wavelength towards which living bodies are highly transmissive.

In order to generate photoacoustic waves effectively it is necessary to emit light over a sufficiently short time in accordance with the thermal characteristics of the object. If the object is a living body, the pulse width of the pulsed light that is generated is preferably about 1 nanosecond to 200 nanoseconds.

In the present embodiment, a single light source is used as the light source 11, but a plurality of light sources may also be used. In that case there may be used a plurality of light sources that oscillate at the same wavelength, or a plurality of light sources that oscillate at different wavelengths.

The irradiation intensity of light irradiated onto the object can be increased when there is used a plurality of light sources that oscillate at the same wavelength. Further, wavelength-dependent differences in an optical characteristic value distribution can be measured when using a plurality of light sources that oscillate at different wavelengths. For instance, wavelength-dependent differences in an optical characteristic value distribution can be measured through the use of a laser that utilizes a dye or an optical parametric oscillator (OPO) capable of variably controlling the wavelength of oscillation.

In the present embodiment, the light source 11 is configured to be capable of emitting two kinds of light i.e. light for measurement and light for decomposing the contrast agent. The light for decomposing the contrast agent may be, for example, light having a wavelength suitable for decomposing the contrast agent, or light of a wavelength identical to that of the light for measurement but of greater pulse width. Different wavelengths and pulse widths may be combined herein. In the explanation of the embodiments, the former will be referred to as measurement light and the latter as decomposition light.

<<Optical System 13>>

The optical system 13 is a means for guiding, to the object 15, light (reference symbol 12) emitted by the light source 11, while bringing the light to a desired light distribution shape by way of an optical component such as a lens or a mirror. The optical system 13 may be configured to allow the light emitted by the light source 11 to propagate, for instance through an optical waveguide such as an optical fiber, and be guided towards the object 15. The optical system 13 may be configured out of, for example, optical components, i.e., a mirror that reflects light, a lens that condenses, expands or alters the shape of light, or a diffusion plate that diffuses light. However, the optical system is not limited thereto, and any optical system may be used in the optical system 13 so long as the light emitted by the light source 11 can be irradiated, with a desired shape, onto the object 15. Although the light may be condensed by a lens, a diagnosis region can be expanded, while securing safety towards the living body, through widening of the surface area of the light by a certain extent. In the present embodiment the light passing through the optical system 13 is expanded by an irradiation unit 30 being the output end, and is thereupon irradiated onto the object.

While the above explanation gives an example of irradiating the object 15 with light and using light diffused in the object, a phase modulator (for example, Spatial Light Modulator (SLM)), as a constituent element of the optical system, may be applied to phase-matched laser light having been expanded by a lens. By modulating phase at each site with SLM, light can be condensed to a certain level even in a diffusible medium such as a living body. Such technique is described in “Universal Optimal Transmission of Light Through Disordered Materials, PHYSICAL REVIEW LETTERS PRL 101, 120601-1-120601-4, 2008”.

<<Object 15>>

The object 15 does not make up the photoacoustic apparatus according to the present embodiment, but will be explained herein for the sake of convenience. The photoacoustic apparatus 1000 according to the present embodiment is an apparatus used for instance for diagnosis of malignant tumors and vascular disease, as well as chemotherapy follow-up, in humans and animals. The object 15, which is typically a living body, is a segment targeted for diagnosis, for instance breasts, fingers, limbs and the like in humans or animals.

In the present embodiment, the light absorber inside the object 15, being the observation target, is classified into a light absorber originally present inside the object and a light absorber that is injected into the object from outside. The former may be for instance oxygenated hemoglobin, reduced hemoglobin, or blood or a blood vessel including the foregoing, while the latter is for instance a contrast agent.

<<Contrast Agent 1012>>

The contrast agent 1012 is mainly a light absorber that is externally administered to the object 15 with a view to improving the contrast (S/N ratio) of a photoacoustic signal distribution. Besides the light absorber itself, a material for controlling in-vivo kinetics may be incorporated into the contrast agent 1012. Examples of materials for in-vivo kinetics control include serum-derived proteins such as albumin or IgG, and water-soluble synthetic polymers such as polyethylene glycol. In the present description, accordingly, the term contrast agent encompasses a light absorber itself, as well as a contrast agent resulting from covalent bonding of a light absorber and another material, and a contrast agent in which a light absorber and another material are held by physical interactions.

When the object 15 is a living body, near-infrared light (wavelength from 600 nm to 900 nm) is preferably used as the irradiation light, from the viewpoint of safety and living body transmissivity. Accordingly, a material having at least light absorption characteristics in the near-infrared wavelength region is used as the contrast agent 1012. Examples include cyanine-based compounds (also referred to as a cyanine dyes) typified by indocyanine green, and inorganic compounds typified by gold or iron oxides.

The molar absorption coefficient of the cyanine-based compound in the present embodiment at an absorption maximum wavelength is preferably 10⁶ M⁻¹cm⁻¹ or higher. Examples of the structure of the cyanine-based compound in the present example include the structures represented by Formulas (1) to (4).

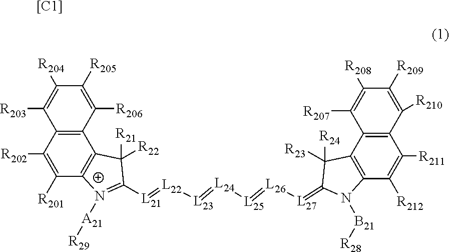

In Formula (1), R₂₀₁ to R₂₁₂ represent each independently a hydrogen atom, a halogen atom, SO₃T₂₀₁, PO₃T₂₀₁, a benzene ring, a thiophene ring, a pyridine ring or a linear or branched alkyl group having 1 to 18 carbon atoms. Further, T₂₀₁ represents any one from among a hydrogen atom, a sodium atom and a potassium atom. In Formula (1), R₂₁ to R₂₄ represent each independently a hydrogen atom or a linear or branched alkyl group having 1 to 18 carbon atoms. In Formula (1), A₂₁ and B₂₁ represent each independently a linear or branched alkylene group having 1 to 18 carbon atoms. In Formula (1), L₂₁ to L₂₇ represent each independently CH or CR₂₅. Herein, R₂₅ represents a linear or branched alkyl group having 1 to 18 carbon atoms, a halogen atom, a benzene ring, a pyridine ring, a benzyl group, ST₂₀₂ or a linear or branched alkylene group having 1 to 18 carbon atoms. Further, T₂₀₂ represents a linear or branched alkyl group having 1 to 18 carbon atoms, a benzene ring or a linear or branched alkylene group having 1 to 18 carbon atoms. In Formula (1), L₂₁ to L₂₇ may form a 4-membered to 6-membered ring. In Formula (1), R₂₈ represents any one of —H, —OCH₃, —NH₂, —OH, —CO₂T₂₈, —S(═O)₂OT₂₈, —P(═O)(OT₂₈)₂, —CONH—CH(CO₂T₂₈)—CH₂(C═O)OT₂₈, —CONH—CH(CO₂T₂₈)—CH₂CH₂ (C═O)OT₂₈ and —OP(═O)(OT₂₈)₂. Further, T₂₈ represents any one from among a hydrogen atom, a sodium atom and a potassium atom. In Formula (1), R₂₉ represents any one of —H, —OCH₃, —NH₂, —OH, —CO₂T₂₉, —S(═O)₂OT₂₉, —P(═O)(OT₂₉)₂, —CONH—CH(CO₂T₂₉)—CH₂(C═O)OT₂₉, —CONH—CH(CO₂T₂₉)—CH₂CH₂(C═O)OT₂₉ and —OP(═O)(OT₂₉)₂. Further, T₂₉ represents any one from among a hydrogen atom, a sodium atom and a potassium atom.

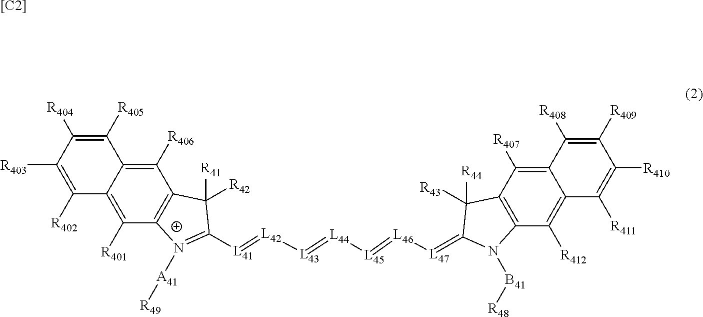

In Formula (2), R₄₀₁ to R₄₁₂ represent each independently a hydrogen atom, a halogen atom, SO₃T₄₀₁, PO₃T₄₀₁, a benzene ring, a thiophene ring, a pyridine ring or a linear or branched alkyl group having 1 to 18 carbon atoms. Further, T₄₀₁ represents any one from among a hydrogen atom, a sodium atom and a potassium atom. In Formula (2), R₄₁ to R₄₄ represent each independently a hydrogen atom or a linear or branched alkyl group having 1 to 18 carbon atoms. In Formula (2), A₄₁ and B₄₁ represent each independently a linear or branched alkylene group having 1 to 18 carbon atoms. In Formula (2), L₄₁ to L₄₇ represent each independently CH or CR₄₅. Herein, R₄₅ represents a linear or branched alkyl group having 1 to 18 carbon atoms, a halogen atom, a benzene ring, a pyridine ring, a benzyl group, ST₄₀₂ or a linear or branched alkylene group having 1 to 18 carbon atoms. Further, T₄₀₂ represents a linear or branched alkyl group having 1 to 18 carbon atoms, a benzene ring or a linear or branched alkylene group having 1 to 18 carbon atoms. In Formula (2), L₄₁ to L₄₇ may form a 4-membered to 6-membered ring. In Formula (2), R₄₈ represents any one of —H, —OCH₃, —NH₂, —OH, —CO₂T₄₈, —S(═O)₂OT₄₈, —P(═O)(OT₄₈)₂, —CONH—CH(CO₂T₄₈)—CH₂ (C═O)OT₄₈, —CONH—CH(CO₂T₄₈)—CH₂CH₂(C═O)OT₄₈ and —OP(═O)(OT₄₈)₂. Further, T₄₈ represents any one from among a hydrogen atom, a sodium atom and a potassium atom. In Formula (2), R₄₉ represents any one of —H, —OCH₃, —NH₂, —OH, —CO₂T₄₉, —S(═O)₂OT₄₉, —P(═O)(OT₄₉)₂, —CONH—CH(CO₂T₄₉)—CH₂(C═O)OT₄₉, —CONH—CH(CO₂T₄₉)—CH₂CH₂(C═O)OT₄₉ and —OP(═O)(OT₄₉)₂. Further, T₄₉ represents any one from among a hydrogen atom, a sodium atom and a potassium atom.

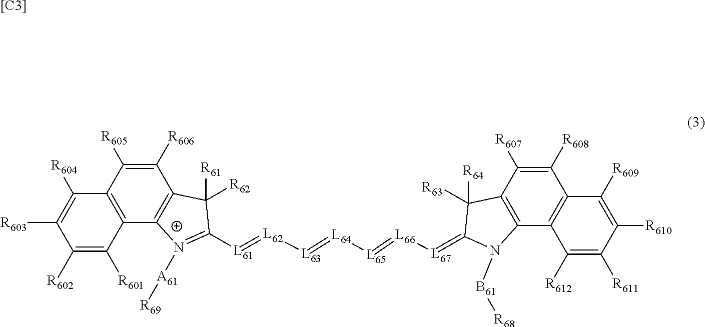

In Formula (3), R₆₀₁ to R₆₁₂ represent each independently a hydrogen atom, a halogen atom, SO₃T₆₀₁, PO₃T₆₀₁, a benzene ring, a thiophene ring, a pyridine ring or a linear or branched alkyl group having 1 to 18 carbon atoms. Further, T₆₀₁ represents any one from among a hydrogen atom, a sodium atom and a potassium atom. In Formula (3), R₆₁ to R₆₄ represent each independently a hydrogen atom or a linear or branched alkyl group having 1 to 18 carbon atoms. In Formula (3), A₆₁ and B₆₁ represent each independently a linear or branched alkylene group having 1 to 18 carbon atoms. In Formula (3), L₆₁ to L₆₇ represent each independently CH or CR₆₅. Herein, R₆₅ represents a linear or branched alkyl group having 1 to 18 carbon atoms, a halogen atom, a benzene ring, a pyridine ring, a benzyl group, ST₆₀₂ or a linear or branched alkylene group having 1 to 18 carbon atoms. Further, T₆₀₂ represents a linear or branched alkyl group having 1 to 18 carbon atoms, a benzene ring or a linear or branched alkylene group having 1 to 18 carbon atoms. In Formula (3), L₆₁ to L₆₇ may form a 4-membered to 6-membered ring. In Formula (3), R₆₈ represents any one of —H, —OCH₃, —NH₂, —OH, —CO₂T₆₈, —S(═O)₂OT₆₈, —P(═O)(OT₆₈)₂, —CONH—CH(CO₂T₆₈)—CH₂ (C═O)OT₆₈, —CONH—CH(CO₂T₆₈)—CH₂CH₂(C═O)OT₆₈ and —OP(═O)(OT₆₈)₂. Further, T₆₈ represents any one from among a hydrogen atom, a sodium atom and a potassium atom. In Formula (3), R₆₉ represents any one of —H, —OCH₃, —NH₂, —OH, —CO₂T₆₉, —S(═O)₂OT₆₉, —P(═O)(OT₆₉)₂, —CONH—CH(CO₂T₆₉)—CH₂ (C═O)OT₆₉, —CONH—CH(CO₂T₆₉)—CH₂CH₂ (C═O)OT₆₉ and —OP(═O)(OT₆₉)₂. Further, T₆₉ represents any one from among a hydrogen atom, a sodium atom and a potassium atom.

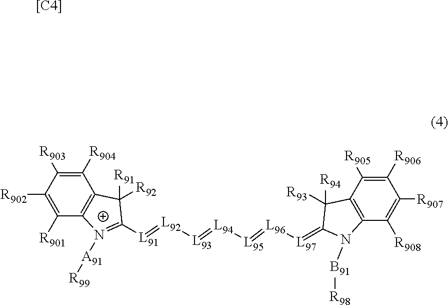

In Formula (4), R₉₀₁ to R₉₀₈ represent each independently a hydrogen atom, a halogen atom, SO₃T₉₀₁, PO₃T₉₀₁, a benzene ring, a thiophene ring, a pyridine ring or a linear or branched alkyl group having 1 to 18 carbon atoms. Further, T₉₀₁ represents any one from among a hydrogen atom, a sodium atom and a potassium atom. In Formula (4), R₉₁ to R₉₄ represent each independently a hydrogen atom or a linear or branched alkyl group having 1 to 18 carbon atoms. In Formula (4), A₉₁ and B₉₁ represent each independently a linear or branched alkylene group having 1 to 18 carbon atoms. In Formula (4), L₉₁ to L₉₇ represent each independently CH or CR₉₅. The above R₉₅ represents a linear or branched alkyl group having 1 to 18 carbon atoms, a halogen atom, a benzene ring, a pyridine ring, a benzyl group, ST₉₀₂ or a linear or branched alkylene group having 1 to 18 carbon atoms. The above T₉₀₂ represents a linear or branched alkyl group having 1 to 18 carbon atoms, a benzene ring or a linear or branched alkylene group having 1 to 18 carbon atoms. In Formula (4), L₉₁ to L₉₇ may form a 4-membered to 6-membered ring. In Formula (4), R₉₈ represents any one of —H, —OCH₃, —NH₂, —OH, —CO₂T₉₈, —S(═O)₂OT₉₈, —P(═O)(OT₉₈)₂, —CONH—CH(CO₂T₉₈)—CH₂(C═O)OT₉₈, —CONH—CH(CO₂T₉₈)—CH₂CH₂ (C═O)OT₉₈ and —OP(═O)(OT₉₈)₂. Further, the above T₉₈ represents any one from among a hydrogen atom, a sodium atom and a potassium atom. In Formula (4), R₉₉ represents any one of —H, —OCH₃, —NH₂, —OH, —CO₂T₉₉, —S(═O)₂OT₉₉, —P(═O)(OT₉₉)₂, —CONH—CH(CO₂T₉₉)—CH₂(C═O)OT₉₉, —CONH—CH(CO₂T₉₉)—CH₂CH₂(C═O)OT₉₉ and —OP(═O)(OT₉₉)₂. Further, T₉₉ represents any one from among a hydrogen atom, a sodium atom and a potassium atom.

Instances of cyanine-based compounds in the present example include indocyanine green, SF-64 having a benzotricarbocyanine structure represented by Chemical formula (1), and compounds represented by Chemical formulas (i) to (v).

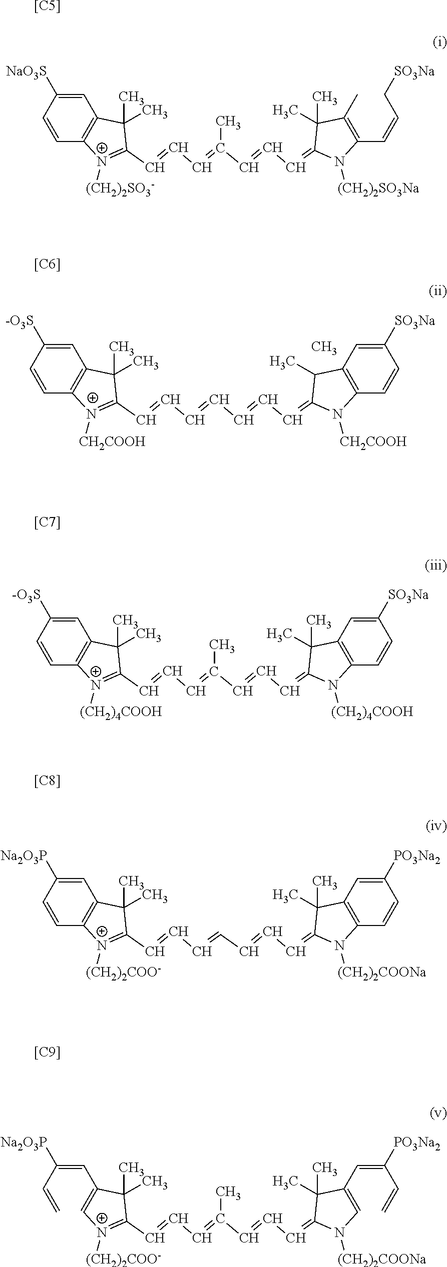

The aromatic rings in the cyanine-based compounds above may be substituted with a sulfonate group, a carboxyl group or a phosphate group. The sulfonate group, carboxyl group or phosphate group may be introduced at a portion other than the aromatic rings.

<<Injection Unit 14>>

The injection unit 14 is a means for injecting the contrast agent from the exterior into the object 15. The injection unit 14 injects the contrast agent 1012 into the object 15 and transmits an injection completion signal to the signal processing unit 19 at the timing where injection is completed. Processing of electrical signals outputted by the probe is initiated on the basis of the above completion signal.

The injection unit 14 may be configured in any way, so long as the latter allows injecting a contrast agent and transmitting, to the signal processing unit 19, the point in time at which the injection operation has been completed. For instance, a known injection system or injector can be used herein.

<<Probe 17>>

The probe 17 is a means for detecting acoustic waves arriving from within the object, and for converting the acoustic waves into an analog electrical signal. The probe is also referred to as an acoustic wave probe or transducer. Any probe may be used herein, for instance a probe relying on piezoelectric phenomena, resonance of light, or changes in capacitance.

The acoustic wave probe may be a one-dimensional or two-dimensional array of a plurality of acoustic wave detection elements (acoustic wave detectors). Through reception of acoustic wave simultaneously at a plurality of positions, it becomes possible to shorten the measurement time and to reduce the influence of for instance object vibration.

Acoustic waves generated by living bodies are typically ultrasonic waves having a frequency in the range of 100 kHz to 100 MHz. Accordingly, elements capable of detecting the above frequency bands are used in the probe 17. Specifically, there can be used for instance transducers that rely on piezoelectric phenomena, transducers that rely on resonance of light, and transducers that rely on changes in capacitance.

Preferably, the acoustic wave probe that is used has high sensitivity and a wide frequency band. Specific examples include for instance piezoelectric elements that utilize lead zirconate titanate (PZT) or the like, capacitive micromachined ultrasonic transducers (CMUTs) and probes that utilize a Fabry-Perot interferometer. The probe is however not limited to the instances enumerated herein, and any probe may be used so long as the latter fulfills the function of a probe.

The probe 17 is configured to be capable of moving with respect to the object 15, the position of the probe 17 being controlled herein by a position control unit 32. By making the probe movable it becomes possible to receive acoustic waves at a plurality of reception positions, and to increase the amount of data that is used in image reconstruction. In the case of a moving probe 17 acoustic waves are ideally received from as many directions as possible with respect to the object. Therefore, the probe 17 is preferably configured to be movable over as wide an area as possible along the surface of the object 15.

The position control unit 32 preferably utilizes a stepping motor that is capable of moving the probe 17 to any position. Preferably, the position control unit 32 moves the probe 17 in such a manner that there varies the relative positional relationship between the probe 17 and the object 15. By doing so it becomes possible to acquire information for obtaining the spatial arrangement (one item of object information) of a sound source (light absorber) inside the object.

For instance, it is conceivable to configure the probe 17 in the form of 128 acoustic wave detection elements, with acoustic waves being received at 120 sites around the object. In this case, the probe 17 acquires an amount of data identical to that of acoustic waves received by acoustic wave detection elements present at a total of 15,360 sites.

The probe 17 may adopt the form of a plurality of acoustic wave detection elements arrayed planarly, but may be configured for instance in the form of a plurality of acoustic wave detection elements arrayed at different positions along a substantially hemispherical surface shape. In this case the acoustic wave detection elements may be disposed in such a manner that the directions of highest reception sensitivity of the elements converge at a given region made up of the center of curvature of the substantially hemispherical surface shape, and the vicinity of the center of curvature.

In the present embodiment, the irradiation unit 30 is provided on the front surface side of the object 15, as illustrated in FIG. 1, and the probe 17 is provided on the back surface side of the object 15, but a configuration may be resorted to in which the probe 17 and the irradiation unit 30 are integrated together. For instance, the probe 17 and the irradiation unit 30 may be provided on the same side with respect to the object 15. Such an implementation will be explained in a fifth embodiment. By doing so it becomes possible to reduce acoustic wave noise (wave component other than acoustic waves generated within the object).

In the present embodiment, the probe 17 is configured to be movable, but the probe is not limited to such a configuration, so long as the positional relationship of the object 15 and the probe 17 can be modified. For instance, only the object 15 may be caused to move while the probe 17 remains immobile; alternatively, both the object 15 and the probe 17 may be caused to move.

The positional relationship between the irradiation unit 30 and the object 15 may be modified, or may be fixed. The irradiation unit 30 appropriately expands light 12 emitted by the light source 11 by way of a lens or the like, not shown, to form irradiation light 34, and irradiate the object 15 with the latter.

<<Signal Collecting Unit 18>>

The signal collecting unit 18 is a means for acquiring an electrical signal transmitted by the probe 17, amplifying the signal, and converting the latter into a digital signal. The signal collecting unit 18 is for instance made up of an amplifier, (operational amplifier or the like), an A/D converter, a field programmable gate array (FPGA) chip or the like. In a case where a plurality of signals are obtained from the probe 17, it is preferable that the plurality of signals can be processed simultaneously. This allows shortening the time required for image reconstruction.

<<Signal Processing Unit 19>>

The signal processing unit 19 is a means (information acquisition unit in the present invention) for processing a signal having undergone digital conversion (hereafter, photoacoustic signal) and reconstructing an image that represents characteristic information on the interior of the object. The signal processing unit 19 is made up of a signal processing module 19a and an image reconstruction module 19b.

The signal processing module 19a is a means for correcting a digital signal using temporal evolution information on the blood concentration of the contrast agent. The image reconstruction module 19b is a means for performing an image reconstruction process on the digital signal after the above correction (corrected digital signal). As a result there is formed image data that denotes characteristic information on the interior of the object 15.

The signal processing unit 19 can be configured for instance in the form of a workstation provided with a processor and a memory. In this case, the functions of the signal processing module 19a and the image reconstruction module 19b may be fulfilled by the workstation. The workstation executes a correction process on the acquired digital signal by means of software programmed beforehand.

The signal processing module 19a, which is interlocked with the injection unit 14, can temporally synchronize the injection operation of the contrast agent 1012, the acquisition of acoustic waves, and changes with time in the blood concentration of the contrast agent 1012. The signal processing module 19a may be configured to perform a noise reduction process on the digital signal acquired from the signal collecting unit 18, and transmit thereafter the resulting signal to the image reconstruction module 19b. The S/N ratio of the object information that is generated can be enhanced thereby.

The image reconstruction module 19b is a means for forming image data by performing an image reconstruction process on the corrected digital signal having been transmitted from the signal processing module 19a. The image reconstruction module 19b performs image reconstruction for instance by back-projection in the time domain or the Fourier domain, as used in tomographic techniques, but other methods may be resorted to herein. Image reconstruction may be carried out by resorting for instance to an inverse problem analysis method by iterative processing, in a case where sufficient time for image reconstruction can be secured. Representative examples of image reconstruction methods used in the image reconstruction module 19b include Fourier transform analysis, universal back-projection, and filtered back-projection.

A probe of focusing type may be used as the probe 17. This allows forming directly image data denoting an optical characteristic distribution of the interior of the object 15 without performing an image reconstruction process such as the above.

The signal processing unit 19 may be configured integrally with the signal collecting unit 18. In this case, image data may be formed as a result of software processing, as performed by the workstation, or may be formed as a result of a hardware process.

<<Input/Output Unit 20>>

The input/output unit 20 is a means for acquiring image data generated by the signal processing unit 19, displaying an image on the basis of the image data, and acquiring inputs from the user. The input/output unit 20 is for instance configured in the form of a touch panel display. The input/output unit 20 may constitute part of the photoacoustic apparatus 1000, or may be provided as an externally attachable unit that is separate from the apparatus 1000.

An outline of the measurement operation performed in the photoacoustic apparatus 1000 will be explained next.

Firstly, pulsed light 12 (measurement light) emitted by the light source 11 passes through the optical system 13, for instance a lens, a mirror, an optical fiber, a diffusion plate or the like, and, while being processed into a desired light distribution shape, is guided onto the object 15 (for instance a cancerous site, a new blood vessel, the face, skin, or a living body), to irradiate the latter.

As the irradiated light propagates through the interior of the object 15, part of the energy of the propagating light becomes absorbed by the non-artificial absorber (blood vessel or the like) 1014, the contrast agent 1012 or the combined absorber 101 in which the foregoing coexist. Which one from among the non-artificial absorber 1014, the contrast agent 1012 and the combined absorber 101 best absorbs the irradiated light depends herein on the wavelength of the light.

Acoustic waves 16 are generated as a result of thermal expansion of the light absorbers as the latter absorb light energy. The acoustic waves propagate through the interior of the object and strike the probe 17.

While moving to an arbitrary reception position around the object 15, the probe 17 receives the acoustic waves propagating from the object 15, and outputs an electrical signal.

The signal collecting unit 18 acquires the electrical signal outputted by the probe 17, performs analog/digital conversion, and outputs the resulting digital signal to the signal processing unit 19. The signal processing unit 19 performs the below-described predetermined process on the outputted digital signal, to form image data for an optical characteristic value, and outputs the image data to the input/output unit 20. The input/output unit 20 displays a viewable image on the basis of the image data. In the explanation below the term “digital signal” denotes a signal generated by the signal collecting unit 18.

The photoacoustic apparatus 1000 according to the present embodiment has three functions: (1) injecting a contrast agent into the object; (2) decomposing the contrast agent using decomposition light; and (3) acquiring information relating to blood flow (a blood flow direction, a blood flow rate). The specific process contents will be explained further on with reference to a flowchart.

Herein there can be separately acquired a signal obtained on the basis of acoustic waves derived from hemoglobin in the blood, being the non-artificial absorber, and a signal obtained on the basis of acoustic waves derived from the contrast agent that is injected from outside. As a result it becomes possible to acquire separately for instance an image derived from hemoglobin in blood, being the non-artificial absorber, and an image derived from the contrast agent.

An image may be generated on the basis of a signal in which the foregoing two signals are mixed. This way, the signal obtained on the basis of the acoustic waves derived from hemoglobin in blood and the signal obtained on the basis of the acoustic waves derived from the contrast agent are added together, and hence an image can be acquired in which there is further enhanced for instance the brightness of a blood vessel portion within the object.

<Process Flow>

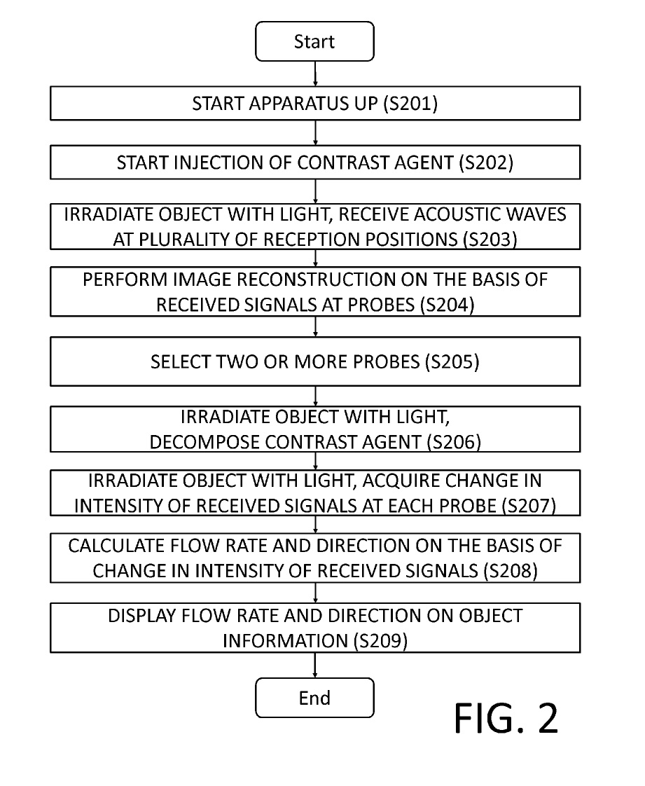

FIG. 2 is a flowchart illustrating a process performed by the photoacoustic apparatus 1000 according to the present embodiment. The process illustrated in FIG. 2 is initiated on the basis of an instruction by the user, after holding of the object 15 by a holding member not shown is complete.

Firstly, supply of power to the photoacoustic apparatus 1000 is initiated, to start the apparatus up (step S201).

Next, the contrast agent 1012 is injected from the injection unit 14 into the object 15 (step S202). For instance, bolus injection may be resorted to as the method for injecting the contrast agent 1012.

Next, measurement light is irradiated onto the object 15, whereupon the probe 17 receives acoustic waves at a plurality of acoustic wave reception positions (step S203).

In step S203, acoustic waves are received while under sequential irradiation of light from the irradiation unit 30, as the probe 17 is caused to move along the object 15. For instance, the probe 17 is caused to move so as to pass predetermined reception positions, with acoustic waves being received for each predetermined reception position. A trajectory of the probe may be set on the basis of information, inputted beforehand, about the predetermined reception position.

The received acoustic waves at each reception position are converted into time-series digital signals, and the latter are temporarily stored in the signal processing unit 19 mapped to respective reception times.

In a case where the object 15 is a living body, acoustic waves derived from the artificial absorber such as the contrast agent and acoustic waves derived from the non-artificial absorber such as hemoglobin can be received individually. For instance, a configuration may be adopted in which acoustic waves derived from the contrast agent injected into the object 15 are received first, and acoustic waves derived from the non-artificial absorber are received next.

Specifically, light of a wavelength that is absorbed mainly by the non-artificial absorber 1014 is irradiated onto the object 15, the generated acoustic waves are received, and thereafter light of a wavelength that is absorbed mainly by the contrast agent 1012 is irradiated onto the object 15, and generated the acoustic waves are received. The content of the process in this case is identical to that where light of a single wavelength is irradiated, but herein light is irradiated a plurality of times upon modification of the wavelength of the irradiated light.

Alternatively, acoustic waves derived from the non-artificial absorber may be received firstly, and acoustic waves derived from both the contrast agent and the non-artificial absorber may be received next, followed by a process for separating the acoustic waves.

Specifically, light is irradiated onto the object 15 at a stage prior to injection of the contrast agent, the generated acoustic waves are received and are stored temporarily in the form of digital signals. The contrast agent is then injected into the object, light is irradiated once more onto the object 15, and the generated acoustic waves are received. Lastly, a difference between the received signals and the signals temporarily stored is acquired. As a result it becomes possible to acquire digital signals substantially corresponding to the acoustic waves derived from the contrast agent. Such a process is made possible through storage of the digital signals and reception times associated with each other.

In the present embodiment, the probe 17 is caused to move to 120 predetermined reception positions in one photoacoustic measurement, with acoustic waves being received at each reception position.

In step S204, the signal processing unit 19 performs an image reconstruction process on the stored digital signals, to form image data. Herein the image data that is formed is three-dimensional voxel data, but the image data may be two-dimensional or one-dimensional data. Further, a configuration may be adopted wherein the user selects which type of image data is to be formed (for instance, one-dimensional, two-dimensional or three-dimensional) image data, and there is formed an image of the selected type.

The digital signal may be reconstructed at each reception position; alternatively, the digital signals may be grouped according to some other criterion, and reconstruction of the digital signal may be then performed for each group. For instance, Fourier transform analysis, universal back-projection, filtered back-projection or sequential reconstruction can be utilized herein as the reconstruction process, but the process is not limited to the foregoing.

Step S205 includes (1) a process of presenting the obtained image data to the user, and receiving an input of a region of interest, and (2) a process of selecting a plurality of acoustic wave detectors that capture photoacoustic waves arriving from the region of interest that has been set.

The region of interest can be acquired for instance through output of the image generated in step S204 to the input/output unit 20, and designation, by the user, of a region of interest on the outputted image.

An acoustic wave detector from among a plurality of acoustic wave detectors can be selected for instance through extraction of an acoustic wave detector such that a contribution ratio thereof with respect to the sum of photoacoustic waves acquired from the region of interest is equal to or higher than a predetermined value. For instance, there may be selected an acoustic wave detector having a signal intensity contribution ratio of 50% or higher.

Alternatively, an acoustic wave detector having a value of signal intensity equal to or greater than a predetermined value may be selected through extraction from a plurality of acoustic wave detectors. For instance, there may be selected an acoustic wave detector that outputs a signal intensity being twice or more the signal intensity outputted by an acoustic wave detector that measures a portion where the object is absent.

Further, a corresponding acoustic wave detector may be selected on the basis of obtained image data and arrangement information of the acoustic wave detectors. For instance, an acoustic wave detector may be selected on the basis of a distance from a point in the region of interest up to the acoustic wave detector.

Moreover, there may be selected an acoustic wave detector positioned in the direction perpendicular to a surface and/or a line being the region of interest.

For example, in the case of measuring a blood vessel the structural arrangement of which has been known, there may be selected an acoustic wave detector positioned in the direction perpendicular to the blood vessel.

Herein, the signal processing unit 19 and the input/output unit 20 function as the region-of-interest setting unit of the present invention.

In step S206, next, the contrast agent is decomposed through irradiation of light (decomposition light) of a wavelength at which the injected contrast agent is decomposed. Decomposition of the contrast agent may be accomplished through irradiation of decomposition light from the light source for measurement, or through irradiation of decomposition light from a dedicated light source provided for decomposition of the contrast agent.

The irradiation time can be set on the basis of the decomposition efficiency of the contrast agent and the detection limit of a change in signal intensity. For instance, the irradiation time can be set so that the change in signal intensity is twice or larger than that of noise.

Ordinarily, the longer the irradiation time is, the further decomposition of the contrast agent is promoted; accordingly, continuous light may be used, instead of pulsed light, as the decomposition light that is irradiated in the present step.

The frequency of the light may be set to be higher than that of the light for measurement.

In a case where the irradiation area of light is movable, there may be irradiated decomposition light just onto the region of interest that has been set.

In step S207, next, measurement light is irradiated again onto the object, and there is acquired a change in the intensity of the received signal in each acoustic wave detector selected in step S205. Acquisition of the change in the intensity of the received signal may be initiated simultaneously with switchover to the process in step S207, or may be initiated through monitoring of the signals outputted by the probes. Acquisition of the change in the intensity of the received signal may be initiated simultaneously with switchover of the process to step S206.

In step S208, there are calculated the flow rate and flow direction of blood in the region of interest. Specifically, a degree of signal recovery is acquired on the basis of the change in the intensity of the received signal for each acoustic wave detector, and the degrees of signal recovery are compared, to identify the inflow side and the outflow side.

In step S206, decomposition light is irradiated onto a predetermined region of the object, and as a result a state is brought about in which no contrast agent is present in blood vessels within that region. Upon discontinuation of irradiation of the decomposition light, blood containing a contrast agent flows in from other segments. That is, the signal derived from the contrast agent recovers sequentially in the order inflow side and outflow side. The direction of blood flow can be estimated as a result on the basis of the degree of recovery of signal intensity.

The flow rate of blood can be calculated using physical structure values for specific tissues. The flow rate of blood may be calculated using a blood vessel diameter inferred on the basis of the image acquired in step S204.

Next, in step S209 the calculated flow rate and flow direction of blood are superimposed on the image data. For instance, the inflow side may be displayed in a warm color and the outflow side in a cool color; alternatively, the blood direction may be displayed in the form of arrows. Display may be accomplished by relying on an ordinary method such as vector mapping.

Optimal contrast agent decomposition conditions and imaging conditions for presenting the flow rate direction may be set through execution of steps S207 and S208 while increasing and reducing the number of laser pulses in step S206. Further, optimal imaging conditions may be set by modifying the imaging conditions in steps S207 and S208, while decomposing the contrast agent in step S206.

In the first embodiment, the flow rate and flow direction of blood can be acquired through temporary decomposition of a contrast agent within a predetermined area, and through acquisition of a change in signal intensity derived from re-inflow of the contrast agent.

### Second Embodiment

The photoacoustic apparatus according to the first embodiment acquires a change in the intensity of received signals in a plurality of acoustic wave detectors, after decomposition of the contrast agent. However, changes in the intensity of the received signals occur not only as a result of the inflow of contrast agent, but also on account of normal pulsations. To address this, in a second embodiment, the object is measured using at least a plurality of wavelengths, and pulsations are corrected using the measurement results.

In the second embodiment, the light source 11 is configured to be capable of emitting a first wavelength at which mainly the absorptivity of the contrast agent is high, and a second wavelength at which the absorptivity of hemoglobin is higher than that at the first wavelength.

In the second embodiment, there is executed beforehand a step of measuring a change in the intensity of the received signal in each acoustic wave detector, using the second wavelength, and identifying a corresponding periodic change. The change in the intensity of the received signals as acquired in step S207 is corrected on the basis of the periodic change acquired using the second wavelength. As a result it becomes possible to eliminate the influence of pulsations and to work out more accurately the flow rate and flow direction of blood.

For instance, the change in the intensity of the received signal in each acoustic wave detector at the second wavelength, may be calculated as a relative ratio, and be multiplied by a coefficient, after which the result is subtracted from the intensity of the received signal in each acoustic wave detector at the first wavelength.

### Third Embodiment

In the first and second embodiments, a region of interest was set by a user on the basis of an image obtained through photoacoustic measurement. In a third embodiment, by contrast, a region of interest is set using an image obtained by another object information acquisition apparatus.

The other object information acquisition apparatus may be for instance an image forming apparatus such as another photoacoustic apparatus, an ultrasound diagnosis apparatus, a magnetic resonance imaging (MRI) apparatus, a computed tomography (CT) apparatus or the like.

In the third embodiment, steps S203 and S204 are replaced by acquisition of the object image by another image forming apparatus, the object image being then used in step S205. In the third embodiment, the object image can be acquired in accordance with an appropriate method according to the imaging target, and thus the setting precision of the region of interest can be increased as a result.

In the case of measuring a blood vessel the structural arrangement of which has been known, there may be selected an acoustic wave detector positioned in the direction perpendicular to the blood vessel.

### Fourth Embodiment

In a fourth embodiment, acoustic waves are received by fixing the probe 17 at a position according to a set region of interest, to acquire changes in signal intensity within the region of interest.

The process of step S207 is performed once the probe 17 has been fixed at a position according to the region of interest; as a result, it becomes possible to reduce the influence on image data and to increase the calculation precision of flow rate and flow direction in the region of interest.

In the present embodiment, a target acoustic wave detector can be selected on the basis of a positional relationship with respect to the region of interest. That is, the processing time in step S205 can be shortened.

In the case of measuring a blood vessel the structural arrangement of which has been known, there may selected an acoustic wave detector positioned in the direction perpendicular to the blood vessel.

### Fifth Embodiment

In the fifth embodiment, acoustic waves are received using a probe in which a plurality of acoustic wave detectors are disposed on a hemispherical holder.

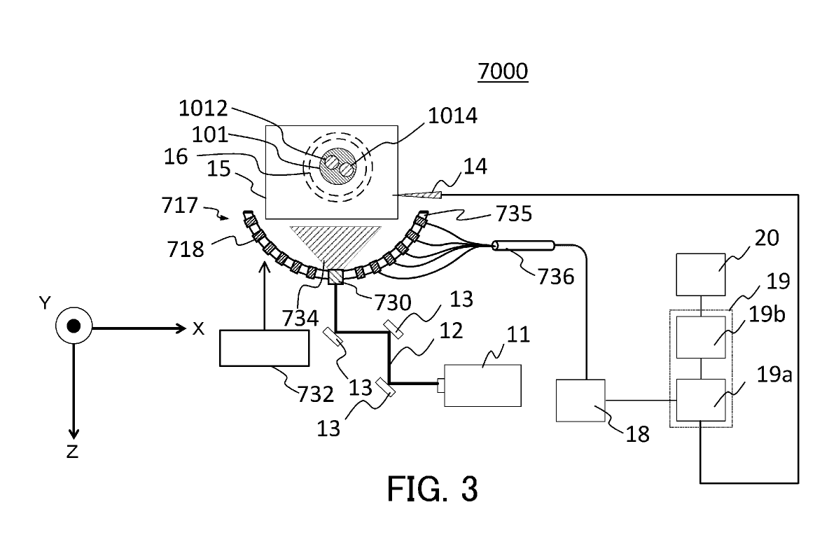

FIG. 3 is a block diagram illustrating a photoacoustic apparatus 7000 according to the fifth embodiment. Reference numerals from 700 to 799 will be used to denote elements similar to those of the first embodiment, with tens and ones digits of the numerals being shared with corresponding elements of the first embodiment. Such identical elements will not be explained unless necessary.

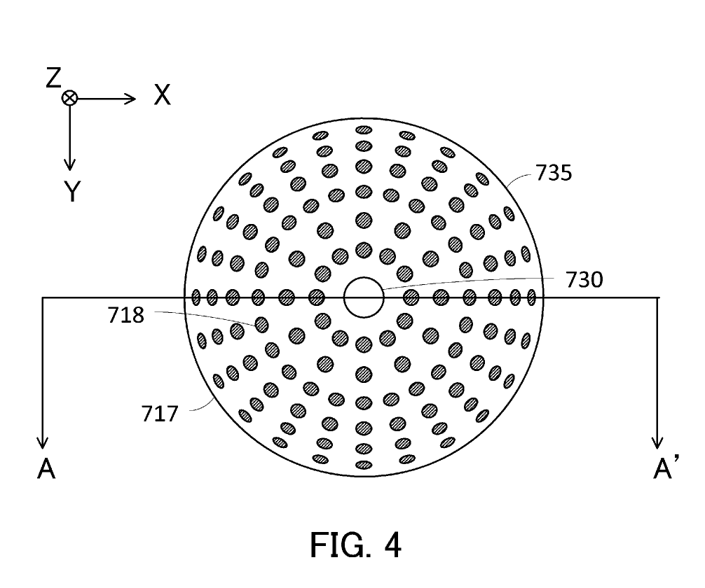

FIG. 4 is a diagram of a probe 717 observed from the Z-axis direction. The probe 717 in FIG. 3 corresponds to the A-A′ cross-section in FIG. 4.

In the fifth embodiment, the probe 717 is made up of a holder 735 and a plurality of acoustic wave detection elements 718 disposed on the holder 735.

The holder 735 is a holding member formed as a bowl shape (substantially hemispherical surface shape), with the plurality of acoustic wave detection elements 718 held along that substantially hemispherical surface shape. The plurality of acoustic wave detection elements 718 are disposed in such a manner that the directions of highest reception sensitivity of the respective elements converge at one point.

In the present embodiment, the acoustic wave detection elements 718 are disposed in such a manner that the directions of highest reception sensitivity of the plurality of acoustic wave detection elements 718 are aimed towards the center of curvature of the holder 735. The electrical signals outputted by the acoustic wave detection elements 718 are combined by a signal line 736 and are outputted to the signal collecting unit 18 via the signal line 736. Subsequent signal processing and so forth are identical to those in the embodiment described above.

In the fifth embodiment, the irradiation unit 730 is disposed at the center of the holder 735. That is, the probe 717 and the irradiation unit 730 are integrated together. The irradiation unit 730 irradiates irradiation light 734 onto the object 15 in an opposite direction to that in the first embodiment. In the first embodiment, specifically, light is irradiated in a direction (Z-axis positive direction) towards the probe 17, whereas in the present embodiment light is irradiated in a direction (Z-axis negative direction) away from the probe 717.

The position control unit 732 controls the position of the probe 717 using a moving mechanism not shown. The position control unit 732 may for instance cause the probe 717 to move spirally within the X-Y plane, and the probe 717 may be configured to be movable in the Z-axis direction.

FIG. 4 is a diagram of the probe 717 and the irradiation unit 730 viewed from the object side. As illustrated in the figure, in the present embodiment, the irradiation unit 730 is disposed at the center, and the acoustic wave detection elements 718 are disposed concentrically, but other arrangements may be adopted. For instance, the acoustic wave detection elements 718 may be arrayed spirally, and the irradiation unit 730 may be disposed at a position other than the center. In the present example, the irradiation unit 730 has a circular shape, but may adopt any shape.

In the fifth embodiment, thus, a plurality of acoustic wave detection elements are disposed three-dimensionally so as to surround the object; as a result, it becomes possible receive efficiently the acoustic waves arriving from the object.

There may be selected an acoustic wave detector positioned in the direction perpendicular to a surface and/or a line being the region of interest, from among a plurality of acoustic wave detection elements arranged so as to surround the object.

For example, in the case of measuring a blood vessel the structural arrangement of which has been known, there may be selected an acoustic wave detector positioned in the direction perpendicular to the blood vessel.

### Sixth Embodiment

In the sixth embodiment, focusing type detectors which focus a reception range of an acoustic wave by means of an acoustic lens, etc. are used as a plurality of acoustic wave detectors disposed on a hemispherical holder.

The sixth embodiment is the same as the fifth embodiment except that the acoustic wave detection elements 718 in FIG. 3 are focusing type detectors.

By using such focusing type detectors, the range from which each detector receives an acoustic wave becomes clear in step S205 of the flowchart in FIG. 2. Namely, it becomes possible to easily select two or more acoustic wave detectors (or probes).

### Seventh Embodiment

In the seventh embodiment, a phase modulator is disposed in the optical system that propagates light, and light is condensed at a specific site in the object, and an acoustic wave is received from the region of the condensed light. For instance, SLM is disposed at the irradiation unit 730 in FIG. 3, and phase is modulated for each site of laser light, and light is condensed only at a specific blood vessel through which a contrast agent passes.

By means of such embodiment, it becomes possible to receive only a signal from the position at which light has been condensed even when the acoustic detection elements 718 can receive an acoustic wave from various directions, and it thereby becomes possible to more accurately acquire a change in intensity of received signals in step S207.

(Variation)

The explanation in the embodiments is illustrative of the present invention, but the latter can be carried out including modifications and combinations, as appropriate, without departing from the gist of the invention.

For instance, the present invention can be realized as a photoacoustic apparatus that includes at least part of the above processes. The invention can also be realized as a method for controlling a photoacoustic apparatus including at least part of the above processes. Further, the invention can be realized in the form of free combinations of the above processes and means, so long as no technical contradictions arise in doing so.

In the explanation of the embodiments, two kinds of light have been used, namely measurement light and decomposition light, but the types of light that are used may be two or more types. For instance, there may be used a plurality of types of measurement light, and there may be used a plurality of types of decomposition light.

In the explanation of the embodiments there are acquired the flow rate and flow direction of blood, but the information to be acquired may be either one of the foregoing.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

### REFERENCE SIGNS

11: light source, 17: probe, 18: signal collecting unit, 19: signal processing unit

The present invention allows acquiring information relating to blood flow in photoacoustic tomography.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-094564, filed on May 10, 2016, which is hereby incorporated by reference herein in its entirety.

What is claimed is:

1. A photoacoustic apparatus, comprising:
a light source that irradiates an object with light;
a plurality of acoustic wave detectors that receive acoustic waves generated from the object due to the light, convert the acoustic waves into an electrical signal, and output the electrical signal; and
an information acquisition unit that acquires information on the interior of the object, on the basis of the electrical signals,
wherein the information acquisition unit acquires, for each of the acoustic wave detectors, a change in the intensity of the electrical signal, after the object that has been injected with a contrast agent is irradiated with light that decomposes the contrast agent, and acquires information relating to blood flow inside the object, on the basis of the change in the intensity of the electrical signal.

2. The photoacoustic apparatus according to claim 1, wherein
the information relating to blood flow is information including at least one of a flow rate and a flow direction of blood.

3. The photoacoustic apparatus according to claim 1, further comprising:
a region-of-interest setting unit that receives a designation of a region of interest for the object;
wherein the information acquisition unit acquires the change in the intensity of the electrical signal by using only an acoustic wave detector corresponding to the region of interest.

4. The photoacoustic apparatus according to claim 3, wherein
the region-of-interest setting unit outputs, in the form of an image, information on the interior of the object as acquired by the information acquisition unit, and receives a designation of a region of interest on the outputted image.

5. The photoacoustic apparatus according to claim 3, wherein
the region-of-interest setting unit acquires information on the interior of the object from an object information acquisition apparatus outside the photoacoustic apparatus, outputs the information in the form of an image, and receives a designation of the region of interest on the outputted image.

6. The photoacoustic apparatus according to claim 1, wherein
the light source can emit light having a first wavelength for generating an acoustic wave within the object, and light having a second wavelength, which is longer than the first wavelength, for decomposing the contrast agent.

7. The photoacoustic apparatus according to claim 1, wherein
the light source can emit pulsed light for generating acoustic waves within the object, and continuous light for decomposing the contrast agent.

8. The photoacoustic apparatus according to claim 1, wherein
the light source can emit light having a first pulse width, for generating an acoustic wave within the object, and light having a second pulse width, which is greater than the first pulse width, for decomposing the contrast agent.

9. A method for controlling a photoacoustic apparatus having a light source that irradiates an object with light, and a plurality of acoustic wave detectors that receive an acoustic wave generated within the object on account of the light, and convert the acoustic wave into an electrical signal, the method comprising:
a first information acquisition step of acquiring information on the interior of the object, on the basis of the electrical signal; and
a second information acquisition step of acquiring, for each of the acoustic wave detectors, a change in the intensity of the electrical signal, after the object that has been injected with a contrast agent is irradiated with light that decomposes the contrast agent, and acquiring information relating to blood flow inside the object, on the basis of a change in the intensity of the electrical signal.

10. The method for controlling a photoacoustic apparatus according to claim 9, wherein
the information relating to blood flow is information including one of a flow rate and a flow direction of blood.

11. The method for controlling a photoacoustic apparatus according to claim 9, further comprising:
a region-of-interest setting step of receiving a designation of a region of interest for the object, wherein in the information acquisition step, the change in the intensity of the electrical signal is acquired using only an acoustic wave detector corresponding to the region of interest.

12. The method for controlling a photoacoustic apparatus according to claim 11, wherein
in the region-of-interest setting step, information on the interior of the object as acquired in the information acquisition step is outputted in the form of an image, and a designation of the region of interest is received on the outputted image.

13. The method for controlling a photoacoustic apparatus according to claim 11, wherein
in the region-of-interest setting step, information on the interior of the object is acquired from an object information acquisition apparatus outside the photoacoustic apparatus, the information is outputted in the form of an image, and a designation of the region of interest is received on the outputted image.
